(19) United States
(12) Patent Application Publication
(10) Pub. No.: US 2008/0058284 A1
(43) Pub. Date: Mar. 6, 2008

(54) Pharmaceutical compositions of semicarbazones and/or thiosemicarbazones and/or their derivatives and products of these compositions and their uses as anticonvulsant, anti-nociceptive and anti-inflammatory agents, and in the angiogenic therapy

(75) Inventors: Ruben Dario Sinisterra Millan, Belo Horizonte (BR); Marcio M. Coelho, Belo Horizonte (BR); Rafael Pinto Vieira, Belo Horizonte (BR); Letica Regina de Souza Teixeira, Belo Horizonte (BR); Maria Carolina Doreto, Vespasiano (BR); Heloisa de Oliveira Beraldo, Belo Horizonte (BR); Silvia Passos Andrade, Belo Horizonte (BR)

Correspondence Address: NIXON & VANDERHYE, PC, 901 NORTH GLEBE ROAD, 11TH FLOOR, ARLINGTON, VA, 22203, US

(73) Assignee: UNIVERSIDALE FEDERAL DE MINAS GERAIS, Belo Horizonte (BR)

(21) Appl. No.: 11/882,086

(22) Filed: Jul. 30, 2007

Related U.S. Application Data
(63) Continuation-in-part of application No. 10/503,735, filed on Mar. 29, 2005.

(30) Foreign Application Priority Data
Feb. 6, 2002 (BR) PI0200751-7

(51) Int. Cl. A61K 31/175; A61K 31/724; A61P 25/08; A61P 29/00

(52) U.S. Cl. 514/58; 514/582; 514/590

(57) ABSTRACT

The present invention is characterized by obtaining inclusion compounds of semicarbazones and/or thiosemicarbazones and/or their derivatives in cyclodextrins and/or their derivatives, which once tested in experimental models of epilepsia allowed the reduction of anticonvulsant dose from 100 mg/kg to 25 mg/kg. This means an increase in bioavailability of compounds in biological systems. Hence inclusion compounds between semicarbazones and/or thiosemicarbazones and cyclodextrins and their derivatives could be new candidates as anticonvulsant agents. The present invention is also characterized by the increase in the anticonvulsant efficacy of the inclusion compounds between cyclodextrins and/or their derivatives and semicarbazones and/or thiosemicarbazones and/or their derivatives in comparison to free components.

### CROSS REFERENCE TO RELATED APPLICATION

This application is a continuation-in-part of application Ser. No. 10/503,735, filed Aug. 6, 2004 as the US national phase of international application PCT/BR03/00018 filed Feb. 5, 2003 which designated the U.S. and which claimed the benefit of Brazilian Application No. PI 0200751-7 filed Feb. 6, 2002, the disclosure of each of which is hereby incorporated by this reference.

### BACKGROUND OF THE INVENTION

The present invention is characterized by the preparation of pharmaceutical compositions of semicarbazone and/or thiosemicarbazone and/or their derivatives using cyclodextrins and/or their derivatives and products obtained by this process for using as anticonvulsants, anti-nociceptives, and anti-inflammatory agents, and in the angiogenic therapy.

Another characteristic of the present invention is the use of semicarbazones and/or thiosemicarbazones and/or their derivatives as anti-nociceptive and anti-inflammatory agents and in the angiogenic therapy, mixed to pharmaceutically acceptable excipients, in solution or in the solid state.

The compounds of the present invention are useful in pharmaceutical compositions with conventional carriers or vehicles, for administration to humans or animals in dosages as tablets, capsules, pills, powders, granules, suppositories, sterile parenteral solutions, sterile parenteral suspensions, sterile non parenteral solutions or sterile non parenteral suspensions, oral solutions or oral suspensions oil-water or water-oil suspensions, emulsions, and the necessary quantity of the semicarbazones and/or thiosemicarbazones and/or their derivatives.

Thiosemicarbazones (FIG. 1, Generic structure of semicarbazones and/or thiosemicarbazones) are compounds with a large range of biological applications, presenting antitumoral, antiviral, antibacterial, antimalarial, antituberculosis, fungicide, anti-HIV and anticonvulsant activities [Beraldo, H.; Gambino, D.; Minireviews in Medicinal Chemistry, 4, 159, 2004, West, D. X.; Padhyé, S. B.; Sonawane, P. B., Structure and Bonding, 76, 1, 1991; Dimmock, J. R., Pandeya, S. N., Quail, J. W., Pugazhenthi, U., Allen, T. M., Kao, G. Y., Balzarini, J., DeClercp, E., Eur. J. Med. Chem., 30, 303, 1995].

Semicarbazones (FIG. 1) are analogues of the above mentioned compounds in which oxygen replaces sulfur. A series of publications reports on the anticonvulsant activity of semicarbazones [Beraldo, H.; Gambino, D.; Minireviews in Medicinal Chemistry, 4, 159-165, 2004; Dimmock, J. R., Pandeya, S. N., Quail, J. W., Pugazhenthi, U., Allen, T. M., Kao, G. Y., Balzarini, J., DeClercq, E., Eur. J. Med. Chem., 30, 303, 1995; Dimmock, J. R.; Sidhu, K. K.; Thayer, R. S.; Mack, P.; Duffy, M. J.; Reid, R. S.; Quail, J. W.; Pugazhenthi, U.; Ong, A.; Bikker, J. A.; Weaver, D. F., J. of Med. Chem., 36, 16, 1993; Dimmock, J. R.; Puthucode, R. N.; Smith, J. M.; Heltherington, M.; Quail, W. J.; Pughazenti, U.; Leshler, T.; Stables, J. P., J. Med. Chem., 39, 3984, 1996]. In particular, compounds derived from arylsemicarbazones present anticonvulsant activity in the central nervous system [Kadaba, P. K.; Lin, Z.; U.S. Pat. No. 5,942,527, 1999; Dimmock, J. R.; Puthucode, R. N.; WO9640628, MX9709311, JP11506109, U.S. Pat. No. 5,741,818, 1997; Fujibayashi, Y.; Yokoyama, A.; U.S. Pat. No. 5,843,400, 1996].

Structural variations can lead to significant modifications of the biological activity of semicarbazones and thiosemicarbazones, and the literature reports studies on structure-activity relationships [West, D. X.; Padhyé, S. B.; Sonawane, P. B., Structure and Bonding, 76, 1, 1991; Kadaba, P. K.; Lin, Z.; U.S. Pat. No. 5,942,527, 1999].

Semicarbazones are stable, can be orally administered [Kadaba, P. K.; Lin, Z.; U.S. Pat. No. 5,942,527, 1999] and proved to be more active as anticonvulsants than phenytoin and phenobarbital, which are the most used drugs in neurologic clinic to treat epilepsies in humans [Dimmock, J. R., WO9406758, 1994]. Additionally, they present none or very low toxicity [Dimmock, J. R.; Puthucode, R. N., WO9640628, MX9709311, JP11506109, U.S. Pat. No. 5,741,818, 1997; Fujibayashi, Y.; Yokoyama, A., U.S. Pat. No. 5,843,400, 1996].

In the State-of-the-Art, it is reported that semicarbazones and thiosemicarbazones present anticonvulsant activity in two experimental models of epilepsy: the subcutaneous pentylenetetrazole (scPTZ) screen and the maximum electroshock (MES) screen [Dimmock, J. R.; Sidhu, K. K.; Thayer, R. S.; Mack, P.; Duffy, M. J.; Reid, R. S.; Quail, J. W.; Pugazhenthi, U.; Ong, A.; Bikker, J. A.; Weaver, D. F., J. of Med. Chem., 36, 16, 1993; Dimmock, J. R.; Pandeya, S. N.; Quail, J. W.; Pugazhenthi, U.; Allen, T. M.; Kao, G. Y.; Balzarini, J.; DeClercq, E., Eur. J. Med. Chem., 30, 303, 1995; Dimmock, J. R.; Sidhu, K. K.; Tumber, S. D.; Basran, S. K.; Chen, M.; Quail, J. W.; Yang, J.; Rozas, I.; Weaver, D. F., Eur. J. Med. Chem., 30, 287, 1995; Dimmock, J. R.; Puthucode, R. N.; Smith, J. M.; Heltherington, M.; Quail, W. J.; Pughazenti, U.; Leshler, T.; Stables, J. P., J. Med. Chem., 39, 3984, 1996; Dimmock, J. R.; Vashishtha, S. C.; Stables, J. P., Eur. J. Med. Chem., 35, 241, 2000; Kadaba, P. K.; Lin, Z., U.S. Pat. No. 5,942,527, 1999; Dimmock, J. R.; Puthucode, R. N., WO9640628, MX9709311, JP11506109, U.S. Pat. No. 5,741,818, 1997; Fujibayashi, Y.; Yokoyama, A., U.S. Pat. No. 5,843,400, 1996].

The existing patents that report the anticonvulsant activity of semicarbazones and thiosemicarbazones are described below.

U.S. Pat. No. 5,942,527 (1999) Kadaba et al. prepared new pharmaceutical formulations containing hydrazones, hydrazines, thiosemicarbazones and semicarbazones and tested the anticonvulsant activity of these compounds in rats with electroshock induced seizures. The compounds showed to be active in oral administrations in doses of 100 mg/Kg and presented low neurotoxicity.

U.S. Pat. No. 5,741,818 (1997), (MX9709311, WO9640628, AU9659938, FI9704447, NO9705663, EP836591, CZ9703874, NZ309707, HU9802637, JP11506109, BR9609408, AU715897, KR99022408) Dimmock et al., prepared semicarbazones derived from 4-phenoxy or 4-phenylthio-benzaldehyde and tested the anticonvulsant activity of these compounds in rats with electroshock induced seizures. The compounds presented no neurotoxicity in doses up to 500 mg/Kg.

WO9406758 (1996) Dimmock, prepared aryl semicarbazones and tested their effect on the central nervous system as anticonvulsants and in the prevention of epileptic seizures. These compounds showed to be more active than phenytoin and phenobarbital in vivo, and than the corresponding semicarbazides. They are stable, can be given orally, and present low or no neurotoxicity.

No pharmaceutical compositions of semicarbazones and/or thiosemicarbazones and/or their derivatives with cyclodextrins and/or their derivatives were found in the State-of-the-Art.

Epilepsy is a morbid condition known for over 3000 years. Due to its incidence and its dramatic manifestations, and its social impact, it has attracted the attention of scholars and laymen.

The World Health Organization (WHO) defines epilepsy as a chronic cerebral disorder with varied etiology characterized by recurring seizures caused by excessive cerebral neuronal discharge. To the present, the pathogenesis of the cerebral disorder is unknown.

The incidence is estimated at about 50 and 120 out of 100,000 people. About 3-5% of the general population will experiment one or more seizures sometime in life [Cockerell, O. C.; Shorvon, S. D.; Epilepsia: Conceitos atuais, Current Medical Literature Ltd. Lemos Editorial e gráficos Ltda. SP, 1997]. There are several frequent types of epilepsy in the population, occurring at any age and sex, most often starting in childhood or adolescence.

Epileptic seizures are clinic event, which reflect either a temporary dysfunction of a small part of the brain (focal seizures) or of a larger area involving the two cerebral hemispheres (generalized seizures).

Epilepsies with identifiable causes (symptomatic) occur in only 30% of the cases and are associated to several disturbs, including infections, traumas, brain tumors, cerebral vascular disease and Alzheimer-Pick disease. Idiopathic epilepsies are transmitted genetically and manifest in certain age groups, and cryptogenic epilepsies are those presumed to have an organic basis, but with unclear etiology.

Epileptic seizures are those, which occur under epileptic conditions and are characterized by motor shaking of some parts of the body (partial seizures) or all the body (generalized seizures.

Non-epileptic convulsive seizures are common symptoms of acute neurologic diseases such as meningitis, cranium encephalic traumas, cerebral vascular diseases and others. Metabolic changes may also be associated to convulsive seizures. Non-organic seizures are those without any pathologic anatomic change correlated to the disturb. Non-organic seizures are most commonly psychogenic (conversion hysterias) Hyperexcitability and synchronism seem to be essential characteristics of the cerebral substrates that can generate a set of neural (neurochemical, neuroanatomic, electrophysiologic, etc.) and behavioral changes [Moraes, M. F. D.; Epilepsia Experimental: estudos eletrofisiológicos e comportamentais em modelos animais de crises convulsivas audiogênicas, Doctorate Thesis presented at Faculdade de Medicina de Ribeirão Preto of Universidade de São Paulo, 1998] that characterize convulsive seizures.

To the present, it has not been possible to establish a simple and practical classification of epilepsies, i.e., of the several chronic diseases whose main symptom is represented by recurring seizures. In contrast, the classification of the different types of convulsive seizures is relatively easy [Goodman and Gilman's, The Pharmacological Basis of Therapeutics, 9^{th} ed., Pergamon Press, New York, 1996]. The classification of epilepsies is based on criteria relative to convulsive seizures, such as frequency, triggering factors, clinical condition, physiopathologic mechanisms, etiology and the age seizures start.

Generalized epileptic seizures are those which occur with loss of conscience and which can either present generalized, bilateral and symmetric motor changes, and vegetative disturbs or not. Absence seizure is generalized and does not have motor manifestation. The responsible neuronal discharge may appear in any area of the brain and may spread to other regions, even involving both cerebral hemispheres.

Among the generalized epileptic seizures distinguishes a convulsing group (tonic-clonic, tonic, clonic, infant spasms, and bilateral myoclonus), and a non-convulsing group (typical absences or petit mal seizures, atypical absences, atonic seizures and akinetic seizures.)

Focal or partial epileptic seizures are those in which electroencephalographic changes are restricted, at least in the beginning, to a specific region of the encephalon. These seizures are classified based on their clinical characteristics as: motor seizures (Jacksonianas, masticatory), sensitive (somatosensitive, cardiocirculatory, respiratory), psychic seizures (delusions, hallucinations) and psychomotor seizures (automatisms).

Treatment is symptomatic, since the drugs available inhibit seizures and there is neither effective prophylaxis nor cure. Keeping to the drug posology is important due to the need of long term treatment with the ensuing side effects of many drugs.

The ideal anticonvulsant drug would suppress all seizures without bringing on any side effects. However, the presently used drugs not only control the convulsant activity in some patients, but also often produce side effects of variable degree, from minimal changes of the CNS to death by aplastic anemia or hepatic insufficiency. It is possible to achieve complete control of seizures in 50% of the patients, and another 25% may improve significantly. Most success is achieved with newly diagnosed patients and it depends on factors such as the type of convulsion, family history, and extent of associated neurological changes [Goodman and Gilman's, The Pharmacological Basis of Therapeutics, 9^{th} ed., Pergamon Press, New York, 1996].

The mechanisms of action of anticonvulsant drugs belong to three different categories. Drugs effective against the most common forms of epileptic convulsions, partial tonic-clonic, and secondarily generalized seizures, seem to result from one of two mechanism. One mechanism reduces the repetitive discharge maintained by a neuron, an effect mediated by the promotion of the inactivity of Na⁺ channels activated by voltage. Another mechanism seems to involve the potentialization of the synaptic inhibition mediated by the γ-aminobutyric acid (GABA), and an intermediate effect through the pre-synaptic action of some drugs and the post-synaptic action of others. The most efficient drugs against a less common form of epileptic convulsion, the absence seizure, lead to the reduction of the activity of the Ca²⁺ channel activated by special voltage, known as T current.

Phenobarbital was the first organic agent synthesized and acknowledged as having anticonvulsant activity. Its sedative properties led investigators to test and demonstrate its efficacy in suppressing convulsive seizures. In a historic discovery, Merrit and Putnam (1938) [Merrit, H. H.; Putnam, T. J.; Arch. Neurol. Psychiatry, 39, 1003, 1938] developed the electroshock convulsive seizure screen in experimental animals to test the anticonvulsant efficacy of chemical agents. They found out from research with a variety of drugs that phenytoin suppressed convulsions without a sedative effect. The electroshock convulsive seizure test is extremely valuable since the drugs efficient against the tonic extension of the hinter legs induced by electroshock are generally effective against partial and tonic-clonic convulsions in humans. Another classification test, induction of convulsive seizures by subcutaneous pentylenetetrazol (sc-PTZ) is useful to identify drugs efficient against absence seizures in humans. Before 1965, the chemical structures of many drugs were rather similar to that of Phenobarbital. These drugs include hydantoins, oxazolydinadiones and succinimides. The agents introduced after 1965 were benzodiazepines (clonazepam and clorazepate), iminostilben (carbamazepine), a carboxylic acid (valproic acid), a phenyltriazine (lamotrigine), and a cyclic analogue to GABA (gabapentin.) [Goodman and Gilman's, The Pharmacological Basis of Therapeutics, 9^{th} ed., Pergamon Press, New York, 1996].

Phenytoin is efficient against all types or partial and tonic-clonic convulsions, but not absence seizures. It is the most extensively studied anticonvulsant agent both in laboratory and in clinical practice. Phenytoin exerts its anticonvulsant action without causing generalized depression of the CNS. In toxic doses, it can provoke excitation signals and a type of decerebration rigidity in lethal levels. The most significant effect of phenytoin is its capacity to change the pattern of convulsions caused by maximum electroshock. It is possible to completely eliminate the characteristic tonic phase; however the residual clonic convulsion can be heightened and prolonged. This modifying action of the convulsion seizure is also observed for other drugs that are efficient against generalized tonic-clonic convulsions. In contrast, phenytoin does not inhibit clonic convulsions induced by pentylenotetrazole. Intravenous administration of phenytoin inhibits convulsion seizures in a susceptible model.

The anticonvulsant use of carbamazepine was approved in the United States in 1974, having being used since the 60's to treat trigeminal nerve neuralgia. It is presently considered a first line drug in the treatment of partial and tonic-clonic convulsions.

The use of valproic acid was approved in the USA in 1978, after being used for over a decade in Europe. The anticonvulsant properties of valproate were discovered serendipitously when it was used as a vehicle for other compounds that were being investigated against convulsions. Valproic acid (n-dipropylacetic acid) is a simple branched chain carboxylic acid.

It is found in the State-of-the-Art that many antiepileptics present anti-nociceptive activity in experimental models and may be useful to aleviate painful conditions in humans. Systemic treatment with lamotrigine, felbamate and gabapentine abolish cold allodynia in a model of chronic constriction in rats [Hunter, J. C.; Gogas, K. R.; Hedley, L. R.; Jacobson, L. O.; Kassotakis, L.; Thompson, J.; Fontana, D. J., Eur. J. Pharmacol. 324, 153-60, 1997]. Per os administration of carbamazepine inhibits the hyperalgesia and edema induce by yeast in rats [Bianchi, M.; Rossoni, G.; Sacerdote, P.; Panerai, A. E.; Berti, F. Eur. J. Pharmacol. 294, 71, 1995]. In addition, it has been demonstrated that the antinociceptive effect induced by antiepileptics results from both peripheral [Todorovic, S. M.; Rastogi, A. J.; Jevtovic-Todorovic, V. Br. J. Pharmacol.; 140, 255, 2003; Carlton, S. M. & Zhou, S. Pain. 76, 201-7, 1998] or central mechanisms [Foong, F. W. & Satoh, M. Br J Pharmacol., 83:493, 1984; Lu, Y. & Westlund, K. N., 1999. J. Pharmacol. Exp. Ther. 290, 214, 1999]. Many antiepileptics that present antinociceptive activity, such as carbamazepine and lamotrigine, also block voltage-sensitive sodium channels [Blackburn-Munro, G.; Ibsen, N.; Erichsen, H. K. Eur J Pharmacol. 445, 231, 2002; Todorovic, S. M.; Rastogi, A. J.; Jevtovic-Todorovic, V., Br. J. Pharmacol., 140, 255, 2003]. Carter et al. have shown that 4-[4-fluorophenoxy]benzaldehyde semicarbazone, a derivative of benzaldehyde semicarbazone (BS), also blocks voltage-sensitive sodium channels [Carter, R. B.; Vanover, K. E.; White, H. S.; Wolf, H. H.; Puthucode, R. N.; Dimmock, J. R. Soc Neurosci Abstr 23, 2163, 1997] and present antinociceptive effect in a model of peripheral neuropathy in rats [Carter, R. B.; Vanover, K. E.; Wielant, W.; Xu Z.; Woodward, R. M.; Ilyin, V. I., In Proceedings, International Symposium “Ion Channels in Pain and Neuroprotection”, Mar. 14-17, 1999, San Francisco, p 19, 1999].

Infection, chemical and physical stimuli, hypoxia, autoimmune reactions, among other endogenous and exogenous factors, may cause cell lesion and, according to the magnitude and duration, may also cause cell death. The presence of these noxious factors induces a local and non-specific response, usually with a protective function, denominated inflammation. This response contributes to eliminate the stimulus that induced the cell lesion and also the necrotic tissue that resulted from this lesion, allowing tissue regeneration [Tracey K. J., Nature, 420, 853, 2002].

One of the symptoms associated with the inflammatory response and also with some pathologic conditions not associated with inflammation that represents the most important cause of suffering for the patients is pain. According to the International Association for the Study of Pain (IASP), pain is defined as an unpleasant experience with sensorial, emotional and cognitive dimensions associated with actual or potential injury.

The detection of noxious stimuli by the neurons is denominated nociception and the neurons that are sensitive to these stimuli are defined as nociceptors. These nociceptors are not usually activated by non-noxious stimuli, as they present a high activation threshold. However, their sensitivity may be increased by inflammation. The cell bodies of the nociceptors are localized in the dorsal root or trigeminal ganglia, according to the region they innervate. These nociceptors make synapse with neurons in the spinal cord dorsal horn or in the brain stem. These secondary neurons project to some structures in the diencephalon, where they make synapse with neurons that project to the cerebral cortex [Woolf, C. J. & Salter, M. W. Science, 288: 1765, 2000].

The sensitization of the nociceptors may result in allodynia and hyperalgesia in the site of the injury or adjacent tissues. The pain may also be reported spontaneously without the need of additional stimuli [Woolf, C. J. & Salter, M. W. Science, 288, 1765, 2000]. The IASP defines hyperalgesia as an exacerbated response to a noxious stimulus and allodynia as pain associated with an innocuous stimulus. These responses are protective mechanisms, as they contribute to behaviour aiming to additional stimulation of the injured site and also to the healing process. The increased responsiveness of the dorsal horn neurons after intense and continuous activation of the nociceptors induces changes of the processing of low and high sensorial stimuli by the central nervous system. Thus, innocuous mechanical stimuli may be interpreted as noxious and may increase the magnitude of pain induced by noxious stimuli [Cervero, F. & Laird, J. M. Pain, 68, 13, 1996]. Some mechanisms involved in the increase of neuronal sensitivity have been identified: increased expression of sodium channels, increased activity of glutamatergic receptors, changes in the effect of gamma-aminobutyric (GABA) on the neuronal excitability and increased calcium influx [Jensen, T. S. Cephalalgia, 21, 765, 2001].

Many of the neuronal changes involved in the pain processing and other manifestations of the inflammatory response may result from the action of a specific group of mediators, the prostaglandins. After the tissue injury, there is a quick induction of the cyclooxygenase (COX) enzyme and the concentrations of eicosanoids, mainly prostaglandins (PG), in the inflammatory exudates are increased. The nonsteroidal anti-inflammatory drugs (NSAIDs) inhibit COX activity and thus the conversion of arachdonic acid to PGG₂ and PGH₂. PGH₂ is substrate to other enzymes that catalyze its conversion to other eicosanoids such as PGD₂, PGE₂, PGI₂, PGF_{2α} and TXA₂ [Bertolini, A.; Ottani, A.; Sandrini, M. Pharmacol. Res., 44, 37, 2001].

The NSAIDs represent a group of different drugs that share some common mechanisms of action. Their analgesic and anti-edematogenic effect result from the inhibition of the synthesis of important inflammatory mediators. Among the NSAIDs there are non-selective (diclofenac, indomethacin and ibuprofen) and COX₂ selective inhibitors (celecoxib, rofecoxib and etoricoxib).

Steroid anti-inflammatory drugs, on the other hand, display a wider inhibitory effect on the production of inflammatory mediators. In addition to inhibiting the production of many eicosanoids, they also inhibit the production of inflammatory cytokines, nitric oxide, adhesion molecules, etc. These drugs present potent anti-inflammatory and immunosupressive activities, justifying their use in the treatment of severe inflammatory conditions such as rheumatoid arthritis, lupus, psoriasis, asthma and anaphylactic shock. Among the most frequent used steroid anti-inflammatory drugs are dexamethasone, prednisone, betamethasone, budesonide and beclomethasone.

Some less conventional drugs have also been used to relieve pain associated with different inflammatory or non-inflammatory conditions. α₂-adrenergic agonists, originally approved as anti-hypertensive drugs, have been used to facilitate the anesthesia, as they present anxiolytic and analgesic activities. They have also been used to alleviate the pain associated with different pathologic conditions when conventional drugs fail [Quan, D. B.; Wandres, D. L.; Schroeder, D. J. Ann. Pharmacother. 27, 313, 1993].

A type of pain, whose relief is not easily attained with the conventional drugs, is that associated with lesions of the brain, spinal cord or peripheral nerves. The neuropathic pain, as it is defined, may occur associated with different forms of cancer, diabetes, amputations, traumatic lesions of nerves, etc. Treatments to reduce the neuronal hyperactivity that characterizes these painful conditions usually provide some relief. Many antiepileptic drugs have been used. Carbamazepine and phenytoin were the first antiepileptics used in the treatment of trigeminal neuralgia, one of the most frequent types of neuropatic pain. Today, other types of neuropathic pain have been shown to be alleviated by antiepileptics and the number of these drugs that also present analgesic activity has been increased. Clinical studies have shown the analgesic efficacy of lamotrigine, gabapentine, pregabaline and topiramate. Valproic acid, thiagabine and felbamate have also been under clinical investigation. The reduction of the neuronal excitability after treatment with these drugs have been attributed to blockade of sodium channels, but other effects may also contribute to their analgesic effect [Jensen, T. S. Cephalalgia 21: 765, 2001].

Although there are different classes of drugs with analgesic activity, there are many painful conditions that are not effectively alleviated by the available drugs.

No pharmaceutical compositions of semicarbazones, thiosemicarbazones and/or their derivatives were found in the State-of-the-Art with anti-nociceptive and/or anti-inflammatory activity, characteristics of the present invention.

Angiogenesis is the biological process of formation of new blood vessels from pre-existing vascular structures. In normal tissues the growing rate of new vessels is kept under rigorous control through the balance of pro and anti angiogenic factors. In injuries, angiogenesis is required not only to ensure maintenance of tissue perfusion but also to allow the increased cellular traffic, which in turn results in reparation. In almost all-medical clinic some degree of angiogenic activity occurs [Folkman, J. Nature Med., 1(1), 27, 1995]. There are fundamental differences between physiological and pathological angiogenesis. Physiological angiogenesis is under stringent control and occurs during embryonic development, endometrial regeneration and wound healing. However, in several pathological conditions, such as solid tumors, rheumatoid arthritis, retinopathy, the process is persistent and deregulated. The newly formed blood vessels in these conditions are usually abnormal (fragile and leaky) leading hemorrhages and local occlusions [Conway, E. M.; Collen, D.; Carmeliet, P. Cardiovasc. Res. 49, 507, 2001]. The identification of pro- or anti-angiogenic compounds has led to the concept of angiogenic therapy that consists of systemic or local application of these compounds in diaseases associated with angiogenesis disorders. [Folkman, J. Nature Med. 1(1), 27 (1995)]. The use of pro-angiogenic factors can improve healing processes in diabetes [Grotendorst, G. R., Martin, G. R., Pencev, D., and cols J. Clin. Invest. 76, 2323 (1985); Greenhalgh, D. V.; J. Traum. Inj. Infect. Critical Care 41, 159, 1996; Folkman, J., Circulation 97, 1108, 1998], heal duodenal ulcers and restore blood flow in ischemic tissues [Folkman, J.; Nature Med. 1(1): 27 1995]. The inhibition of angiogenesis would be a valid drug target for chronic inflammatory diseases such as rheumatoid arthritis as well as for anti-neoplastic therapy [Peacock, D. J., Banquerigo, M. L., Brahn, E., J. Exp. Med. 175, 1135, 1992; Paulus, H. E., Am. Inter. Med. 122, 147, 1995]. To date, over three hundred compounds with pro- or antiangiogenic activities have been described but the advances in their use in the clinic remains at a very early stage.

It is known in the State-of-the-Art that various attempts have been made to identify pro- or anti-angiogenic compounds within the range of clinically available compounds for different medical conditions. The advantage of this approach is that these drugs have already been tested for toxicity and side effects. Imidazole compounds such as carboxyamidotriazole, clotrimazole, econazol clinically used for more than 20 years antifungal agents, have previously been shown to possess antiinflammatory, antiproliferative and antiangiogenic actions in various experimental in vivo models [Benzaquen, L. R., Brugnara, C., Byers, H. R., Gattom-Celli, S., Halperin, J. A., Nature Med, 1(6) 534, 1995; Rocha e Silva M., Belo, A. V., Machado R. D. P., Andrade, S. P., Inflammation, 22, 643, 1998.]. Matter [Matter, A.; Drug Discov. Today 6, 1005, 2001] suggested the division of antiangiogenic compounds into two major categories: vasculotoxins, agents that use vessels components as targets of toxic principles and vasculostatics those that interfere with the process of blood vessel formation. In this category some are classified as low molecular weight compounds.

It is also found in the State-of-the-Art that semicarbazones, thiosemicarbazones and their derivatives are compounds that present a wide range of pharmacological actions, including antifungal and antibacterial. Such effects have been attributed to their antiproliferative activities on microorganisms. However, the effects of semicarbazones and/or thiosemicarbazones and/or their derivatives as anti-inflammatory agents and/or in the angiogenic therapy have not been found in the State-of-the-Art. These effects are the object of the present invention.

To study the mechanisms and the physiological consequences of epilepsy and also the action of anticonvulsant mechanisms, chronic or acute experimental models were used. The most used models for this purpose are the genetic, maximum and minimum electroshock, and chemical model.

In the electroshock model, the epileptic seizures are induced by electric currents from electrodes placed on the head of an animal. Browning (1995) [Browning, R. A., Anatomy of generalized convulsive seizures, in Idiopathic generalized epilepsies. Clinical, experimental and genetic aspects, A. Malafosse, P et al (Eds.), John Libbey & Company Ltd., 1994]. The literature reports that depending on the cerebral region where the current is applied, different types of seizures can be obtained. With trans-auricular electrodes, it was possible to obtain generalized tonic-clonic seizure and with trans-corneal electrodes, limbic seizures.

In genetic models, two combined factors are necessary to obtain a seizure. First, a specific genetic predisposition whose origin is in an anomaly in the neurotransmitters associated with the cholinergic, catecholaminergic, serotoninergic systems and/or amino acids [Jobe, P. C.; Laird, H. E.; Biochem. Pharmacol, 30, 3137, 1981]. The second factor, also called trigger, includes environmental stimuli such as intermittent light, sound, hyperthermia, postural changes and/or new circumstances. Endogenous neurochemical alterations or a hormonal unbalance can also work as triggers. Therefore, for the onset of an epileptic seizure in the genetic model, an inborn predisposition is necessary to seizure together with one or more either exogenous or endogenous triggers. An individual may never have a seizure due to the lack of predisposition or trigger(s) [Aicardi, J.; Course and prognosis of certain childhood epilepsies with predominantly myoclonic seizures and Wada, J. A.; Penry, J. K. and cols; Advances in epileptology, The X^{th} Epilepsy International Symposium. New York; Ravem, 159, 1980].

Audiogenic epilepsy in rats is a genetic model in which seizures are induced by high intensity acoustic stimuli. Four rat colonies with this characteristic were selected. A line derived from Wistar, called WAR-Wistar Audiogenic Rats was bred in Brazil at the laboratory of Neurophysiology and Experimental Neurology of the Physiology department of Faculdade de Medicina de Ribeirao Preto, Universidade de Sao Paulo [Garcia-Cairasco, N.; Doretto, M. C.; Lobo, R. B., Epilepsia, 31, 815, 1990]. A breed of this line is kept at the breeding facilities of Departmento de Fisiologia e Biofísica of Instituto de Ciências Biológicas, Universidade Federal de Minas Gerais, Belo Horizonte [Doretto, M. C.; Oliveira-e-Silva, M.; Ferreira, M.; Garcia-Cairasco, N.; Reis, A. M., Proceedings Congresso Latinoamericano de Epilepsia, Santiago, Chile, 2000]. In WARs, seizures are characterized by running, jumping, atonic falls, tonic convulsions, partial and generalized tonic-clonic convulsions, and clonic spasms [Garcia-Cairasco, N.; Sabbatini, R. M. E., Braz. J. Med. Biol. Res., 16, 171, 1983; Garcia-Cairasco, N.; Doretto, M. C.; Prado, P.; Jorge, B. P. D.; Terra, V. C.; Oliveira, J. A. C., Behav. Brain Res., 58, 57, 1992].

To study the mechanisms and physiological consequences of nociception, inflammation and also the mechanism of action of anti-nociceptive, anti-inflammatory and anti or pro-angiogenic agents, acute or chronic experimental models are used.

To evaluate the antinociceptive and antiedematogenic effect, some experimental models have been used: licking behaviour induced by injection of formaldehyde, thermal allodynia and edema induced by carrageenan and nociceptive response in the hot plate.

In the first model, licking behaviour induced by formaldehyde (0.92%, 20 μL), the inflammatory stimulus is injected into the dorsum of the right hindpaw of mice. Immediately after the injection, the animals present a nociceptive behaviour characterized by licking and biting the injected paw. The licking time is determined between 0 and 5 min and 15 and 30 min after formaldehyde injection. The first phase results from direct activations of primary afferent fibers and is inhibited mainly by centrally acting drugs such as the opioid analgesics. The second phase is associated with the development of an inflammatory response and facilitation of synaptic transmission in the spinal cord. This phase is markedly inhibited by anti-inflammatory drugs, including the steroid and non-steroid anti-inflammatory drugs, bradykinin receptors antagonists, inhibitors of nitric oxide synthesis, etc.

In the model of thermal allodynia, the right hindpaw withdrawal latency to a thermal stimulus (Hargreaves apparatus—Ugo Basile, Italy) is determined in rats before the administration of any drug. The intensity of the thermal stimulus is adjusted to induce a basal latency about 12 s. After the basal lecture, carrageenan (1%, 50 μL) is injected into the right hindpaw. This inflammatory stimulus reduces the latency for paw withdrawal induced by the thermal stimulus, which is characterized as thermal allodynia.

In the hot-plate model, the mice are placed over a hot-plate (57° C.). The latency to jump or lick the paws is determined. In this model, direct and immediate activation of peripheral afferent fibbers occurs. Thus, drugs that act mainly in the central nervous system inhibit the nociceptive response in this model.

To evaluate if a coupound presents antiedematogenic activity, the model of carrageenan-induced edema in rats was used. In this model, before the administration of any drug, the right hindpaw volume is determined with a plethysmomether. After the division of the groups, carrageenan (1%, 50 μL) is injected into the plantar surface. At different times after the injection of carrageenan, the paw volume is determined again. This inflammatory stimulus induces a marked edema that is inhibited by different anti-inflammatory drugs (steroid and non-steroid), inhibitors of nitric oxide synthesis and antagonists of histamine, 5-hydroxytryptamine and bradykinin, inhibitors of nitric oxide syntheses, etc.

The discovery of the importance of the angiogenesis mechanism in several pathologic processes addressed the need of the development of biological in vitro and in vivo models that would be able to evaluate the effectiveness of pro- and anti-angiogenic compounds. The in vitro models are based on culture of endothelial cells from human or experimental animal tissues. The classical assays for angiogenesis in vivo include the hamster cheek pouch, the rabbit ear chamber, the chick chorioallantoic membrane. The use of synthetic matrices implanted subcutaneously into experimental animals have become popular because they induced the growth of a fibrovascular tissue similar to that observed in chronic inflammatory processes or wound healing. Various techniques can be used to quantify blood vessel formation and cells associated with the process. The rate of diffusion of radioactive or fluorescent markers applied locally indicates the degree of vascularization in the site [Andrade, S. P.; Fan, T. P. D., Lewis, G. P. Int. J. Exp. Path. 73, 503, 1987; Andrade, S. P. Machado, R. D. P., Teixeira, A. S., and cols. Microvasc. Res. 54, 253, 1997]. The vascular index has also been determined by extracting from the implants substances injected in the systemic circulation (carmine, Evans Blue) or by extracting blood vessels components (hemoglobin, colagen, laminin) [Maragoudakis, M. E., Panoutsacopoulou, M., Sarmonica, M., Tissue and Cell 20, 531, 1988; Plunkett, M. L., Hailey, J. A. Lab Invest. 62, 510, 1990]. Furthermore, the techniques to measure morphometric parameters include light or electron microscopy of tissue sections stained for endothelial cells or basement membrane. [Weringer, E. J., Kelso, J. M., Tamai, I. Y., and cols. Acta Endocrinol. 99, 101, 1982]. The implants' models with subcutaneous sponges are easy to use, low cost, reproducible and they also allow following the experiment for long period of time (hemoglobin, cell culture) and in vivo (experimental animals).

A drug can be chemically modified to alter its properties such as bio-distribution, pharmacokinetics and solubility. Several methods have been used to increase drug solubility and stability, including organic solvents, emulsions, liposomes, pH adjustments, chemical modifications and complexation of drugs with appropriate encapsulating agents such as cyclodextrins.

Cyclodextrins are cyclic oligosaccharides with six, seven or eight glucopyranose units. Due to steric interactions, cyclodextrins form a cyclic structure shaped like a truncated cone with an apolar internal cavity. They are chemically stable compounds which can be regioselectively modified. Cyclodextrins (hosts) form complexes with several hydrophobic molecules (guests), including guest molecules either completely or partially into the cavity. Cyclodextrins have been used to solubilize and encapsulate drugs, perfumes and flavors as described in the literature [Szejtli, J., Che. Rev., 98, 1743, 1998; Szejtli, J., J. Mater. Chem., 7, 575, 1997]. In respect to detailed toxicity, mutagenicity, teratogenicity and carcinogenicity studies, cyclodextrins present low toxicity [Rajewski, R. A.; Stella, V.; J. Pharm. Sci., 85, 1142, 1996], particularly hydroxylpropyl-β-cyclodextrin [Szejtli, J. Cyclodextrins: Properties and applications. Drug Investig., 2(suppl. 4):11, 1990]. Except for some cyclodextrin derivatives, which provoke damage to erythrocytes in high concentrations, these products in general are not hazardous. The use of cyclodextrins as food additives has been authorized in countries like Japan and Hungary, and for more specific uses in France, and Denmark. In addition, they are obtained from a renewable source from starch degradation. All these characteristics are added reasons for the discovery of new applications. The molecular structure of cyclodextrins is a truncated cone with approximate Cn symmetry. The primary hydroxyls are located on the narrow side of the cone, and the secondary hydroxyls on the broad side. Despite the stability due to the intramolecular hydrogen bonds, it is flexible enough to allow considerable shape modifications.

Cyclodextrins are moderately soluble in water, methanol, and ethanol, and readily soluble in aprotic apolar solvents such as dimethyl sulfoxide, dimethylformamide, N,N-dimethylacetamide and pyridine.

There are many works in State-of-the-art on the effects of the increase in solubility of low soluble guests through inclusion into cyclodextrins. The physical-chemical characteristics and stability of inclusion compounds are well described. [Szejtli, J., Chem. Rev., 98, 1743, 1998; Szejtli, J., J. Mater. Chem., 7, 575, 1997].

The development of new pharmaceutical formulations tends to modify the present concept of drug in the short term. Thus, recently several systems were developed to administer drugs with the purpose of modeling release kinetics, improving drug absorption and stability, or targeting them to specific cellular populations. As a result appear polymeric compositions, cyclodextrins, liposomes, emulsions, multiple emulsions, which serve as carriers of active principles. These compositions can be administered via intramuscular, intravenous, or subcutaneous injection, orally, inhalation, or with implanted or injected devices.

### SUMMARY OF THE INVENTION

The present invention is characterized by obtaining inclusion compounds of semicarbazones and/or thiosemicarbazones and/or their derivatives in cyclodextrins and/or their derivatives, which once tested in experimental models of epilepsia allowed the reduction of anticonvulsant dose from 100 mg/kg to 25 mg/kg. This means an increase in bioavailability of compounds in biological systems. Hence inclusion compounds between semicarbazones and/or thiosemicarbazones and cyclodextrins and their derivatives could be new candidates as anticonvulsant agents.

The present invention is also characterized by the increase in the anticonvulsant efficacy of the inclusion compounds between cyclodextrins and/or their derivatives and semicarbazones and/or thiosemicarbazones and/or their derivatives in comparison to free components.

The present invention is characterized by the efficacy of semicarbazones and/or thiosemicarbazones and/or their derivatives, mixed to pharmaceutically acceptable excipients in solution or in the solid state, as anti-nociceptive and/or anti-inflammatory agents, as non-limiting example benzaldehyde semicarbazone.

Another characteristic of the present invention is the use of semicarbazones and/or thiosemicarbazones and/or their derivatives, mixed to pharmaceutically acceptable excipients in solution or in the solid state, as non-limiting examples benzaldehyde semicarbazone and bromobenzaldehyde semicarbazone in the angiogenic therapy.

The present invention is characterized by the peripheral and central anti-nociceptive actions of semicarbazones and/or thiosemicarbazones and/or their derivatives, mixed to pharmaceutically acceptable excipients in solution or in the solid state, as non-limiting example benzaldehyde semicarbazone.

The present invention is characterized by the efficacy of semicarbazones and/or thiosemicarbazones and/or their derivatives, mixed to pharmaceutically acceptable excipients in solution or in the solid state, as non-limiting example benzaldehyde semicarbazone, on pain relief and edema reduction, which are manifestations that follow many inflammatory conditions, as well as on the relief of naturopathic pain that may result from nerve lesions.

The present invention is also characterized by the anti-angiogenic effect of semicarbazones and/or thiosemicarbazones and/or their derivatives mixed to pharmaceutically acceptable excipients in solution or in the solid state, as non limiting example benzaldehyde semicarbazone, and the pharmaceutical compositions BS/HP-β-CD and BSβ-CD which when tested in the angiogenesis experimental model, (sub-cutaneous sponge implants in Swiss mice anesthetized), inhibited the hemoglobin content (vascular index) in more than 50%, suggesting an anti-angiogenic effect yet not described in the State-of-the-art, as novel anti-angiogenic candidates.

The present invention is characterized by the pro-angiogenic effect of semicarbazones and/or thiosemicarbazones and/or their derivatives, mixed to pharmaceutically acceptable excipients in solution or in the solid state, as non-limiting example bromobenzaldehyde semicarbazone, which when tested in the angiogenesis experimental model (sub-cutaneous sponge implants in Swiss mice anesthetized), increased the hemoglobin content (vascular index) in more than 30%, suggesting a pro-angiogenic effect yet not described in the State-of-the-Art, as novel pro-angiogenic candidates.

### BRIEF DESCRIPTION OF THE DRAWINGS

FIG. 1 illustrates generic structure of semicarbazones and/or thiosemicarbazones.

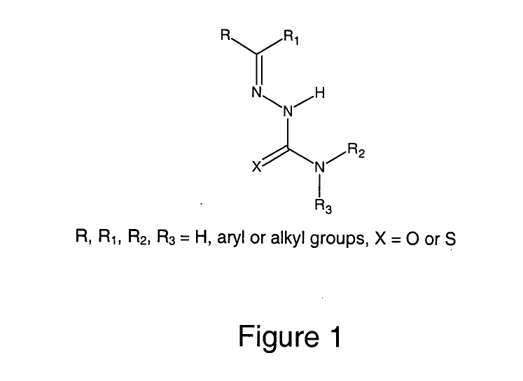

### DETAILED DESCRIPTION OF THE INVENTION

The present invention can be better understood through the following non-limiting examples:

### EXAMPLE 1

### Preparation of Inclusion Compounds Between Hydroxypropyl-β-Cyclodextrin (HP-β-CD) and Semicarbazones and/or Thiosemicarbazones and/or Their Derivatives, Using as Non-Limiting Example Benzaldehyde Semicarbazone

The inclusion compound with hidroxypropyl-β-cyclodextrin (BS/HP-β-CD) was prepared by mixing benzaldehyde semicarbazone (BS) and HP-β-CD in water in 1:1 molar ratio with stirring for 24 hours. The suspension was submitted to a freeze-drying process during 48 hours. A physical mixture (PM) of the same BS:HP-β-CD molar ratio was obtained for comparison.

In the spectrum of HP-β-CD the absorptions at 3425 cm⁻¹, 2920 cm⁻¹, 1650 cm⁻¹ and 1030 cm⁻¹ were attributed to ν(OH), ν(C—H), δ(O—H) and ν(C—O—C) respectively. In the spectrum of BS the absorptions at 3463 cm⁻¹, 3339 cm⁻¹ and 1600 cm⁻¹ were attributed to ν(N—H), ν(NH2) and ν(C═N) respectively [Mattos, S. V. M, Oliveira, L. F. C., Nascimento, A. A. M., Demicheli, C. P., Sinisterra, R. D., Appl. Organometal. Chem., 14, 507 (2000)].

The spectrum of free BS shows absorptions at 3463 and 3339 cm⁻¹ attributed to the ν(N—H) and ν(NH₂) stretching vibrations [Nakamoto, K., Infrared and Raman Spectra of Inorganic and Coordination Compounds, 4^{th} Ed. John Wiley 5& Sons, New York, (1992)]. The ν(C—H) bands of BS were observed in the 2900-3100 cm⁻¹ range. [Nakamoto, K., Infrared and Raman Spectra of Inorganic and Coordination Compounds, 4^{th} Ed. John Wiley 5& Sons, New York, (1992)] Two absorptions attributed to ν(C═O) were found at 1690 and 1650 cm⁻¹ and the maximum at 1600 cm⁻¹ is attributed to ν(C═N) [Kolb, V. M.; Stupar, J. M.; Janota, T. E.; Duax, W. L., J Org. Chem., 54, 2341 (1989)]

In the spectrum of the PM the ν(N—H), ν(NH2) and ν(C—H) absorptions do not appear separately but lay underneath the ν(OH) envelope centered at 3400 cm⁻¹. Also, the intensity of the ν(C═N) absorption and that of ν(C═O) at 1650 cm⁻¹ decrease whereas the intensity of the ν(C═O) absorption at 1690 cm⁻¹ remains practically unchanged [Nakamoto, K., Infrared and Raman Spectra of Inorganic and Coordination Compounds, 4^{th} Ed. John Wiley 5& Sons, New York, (1992)], suggesting some hydrogen bonding between BS and HP-β-CD in the PM.

The same absorption (broad) is observed at 3400 cm⁻¹ in the inclusion compound and the intensities of the two ν(C═O) maxima as well as that of ν(C═N) undergo a substantial decrease with concomitant modification in the intensity ratio, indicating the formation of a new species.

Higher thermal stability was observed for BS after host-guest interaction. The TG curve of BS/HP-β-CD presents a plateau until 300° C. when decomposition occurs, as evidenced by its DTG curve. The TG/DTG curves for HP-β-CD show a weight loss of 6.6% in the 33-122° C. range, associated to the release of five water molecules and reach a plateau of stability until 350° C. when decomposition occurs. BS undergoes decomposition at 256° C. The TG/DTG curves of PM exhibit two decomposition peaks, associated to HP-β-CD and BS.

For HP-β-CD, DSC measurements show one endothermic peak at 52.7° C. corresponding to the release of water and two exothermic peaks at 310.2° C. and 371.4° C., corresponding to the decomposition of the molecule. The DSC curve of the inclusion compound BS/HP-β-CD exhibits one endothermic peak at 52.3° C. attributable to the release of water molecules. Interestingly, the fusion of BS is not observable indicating the interaction of BS and the CD cavity. Moreover, the DSC curve of PM shows approximately the same thermal behavior, suggesting that some inclusion is already observed.

The BS XRD powder diffraction pattern shows sharp peaks at 7.7, 22.7, 23.2, 24.5 and 28.8 2θ, characteristic of a crystalline compound In contrast, HP-β-CD is amorphous. The XRD pattern of PM and of the inclusion compound as compared to that of free HP-β-CD suggest the formation of a higher organized system upon inclusion or association.

NMR spectroscopy provided strong support for the formation of a host-guest complex between BS and HP-β-CD. In free BS the hydrogen relaxation times T₁ for H1, H2, H2′ were determined in the 1.56-1.65 s range and those for H3 and H3′ were 1.60 and 1.69 s respectively. In addition, the measured T₁ for H5, H6 and H7 were 0.93, 0.83 and 0.33 s respectively. Upon inclusion, the values of T₁ of H1, H2 and H2′ shifted to 1.38-1.42 s, T₁ of H3 and H3′ to 1.40 and 1.46 s respectively and T₁ of H5, H6 and H7 to 0.83, 0.73 and 0.29 s respectively. Upon host-guest interaction the values of hydrogen relaxation times T₁ decrease suggesting greater rigidity of the guest's hydrogens. This effect is more pronounced for the aromatic hydrogens, indicating recognition of the phenyl moiety by the CD cavity. The variations observed in T₁ for the semicarbazone moiety could be due to hydrogen bonding between the semicarbazone hydrogens and the hydoxyl groups of the hydroxypropyl substituent on the cyclodextrin.

The signals in the spectra of BS and HP-β-CD are in agreement with data reported in the literature.

Upon inclusion, all hydrogen and carbon signals shift to lower frequencies in agreement with recognition of the phenyl group by the CD cavity, as suggested by the T₁ measurements. Interestingly, the resonance signals of the semicarbazone moiety of BS are also affected ie. NH2 (Δ=0.126), N—H (Δ=0.084), C—H (Δ=0.063) and C═O (Δ=0.238), probably due hydrogen bonding to the hydroxyl groups of the HP-β-CD.

### EXAMPLE 2

### Preparation of Inclusion Compounds Between β-Cyclodextrin and Semicarbazones and/or Thiosemicarbazones and/or Their Derivatives, Using as Non-Limiting Example Benzaldehyde Semicarbazone

The inclusion compound with β-cyclodextrin (BS/β-CD) was prepared by mixing BS and β-CD in water in 1:1 molar ratio with stirring for 48 hours. The suspension was submitted to a freeze-drying process (Labconco Freezone model 177) during 72 hours. A 1:1 BS:β-CD physical mixture was obtained for comparison. The 1:1 BS:β-CD molar ratio in the inclusion compound was confirmed by the Higuchi and Connors method, measuring the BS absorbance at 280 nm in water with a 1 cm path length quartz cell.

As in the case of the inclusion compound BS/HP-β-CD, the first evidence for host-guest interaction was obtained from the modification of the infrared absorptions of BS and β-CD upon inclusion. In the FTIR spectrum of β-CD the absorptions at 3400 cm⁻¹, 2925 cm⁻¹, 1640 cm⁻¹ and 1025 cm⁻¹ were attributed to ν(OH), ν(C—H), δ(O—H) and ν(C—O—C) respectively. In the spectrum of BS the absorptions at 3463 cm⁻¹, 3395 cm⁻¹ and 1600 cm⁻¹ were attributed to ν(N—H), ν(NH₂) and ν(C═N) respectively. The ν(C—H) bands of BS were observed in the 2900-3100 cm⁻¹ range. Two absorptions attributed to ν(C═O) were found at 1690 and 1650 cm⁻¹.

Comparison between the FTIR spectra of BS, the BS/β-CD inclusion compound and the physical mixture reveal important changes upon inclusion. The BS ν(N—H) and ν(NH₂) bands at 3463 cm⁻¹, and 3395 cm⁻¹ respectively were also observed in the spectrum of the physical mixture and in that of the inclusion compound. However, a narrowing of the β-CD absorptions was observed in the inclusion compound, probably due to the breaking of hydrogen bonds upon host-guest interaction. Besides, the intensities of ν(C═O) at 1690 cm⁻¹ and ν(C═N) at 1600 cm⁻¹ of BS undergo a substantial decrease in the spectrum of the inclusion compound which is not observed in the spectrum of the physical mixture, indicating molecular recognition of BS by the β-CD cavity. Crystal structure determinations of BS showed that the distance between the carbonyl carbon and the center of the aryl ring is 9.5 Å On the other hand it is well established that the length distance of β-CD is 7.9 Å, indicating that the cavity could accommodate the aromatic ring as well as part of the BS semicarbazone moiety.

The TG/DTG and DSC curves for β-CD and BS present thermal behaviors as related in the literature.

The TG/DTG curves of the physical mixture exhibit thermal profiles associated to β-CD and BS. The DSC curve shows four endothermic peaks at 70.6° C., 214.7° C., 306.3° C. and 326.3° C., corresponding to β-CD and BS thermal phenomena. The last two peaks, attributed to melting and caramelization of β-CD are observed separately, in contrast to the DSC curve of β-CD, which shows only one thermal event.

The thermal behavior of the BS/β-CD inclusion compound is entirely different. Its TG curve presents a weight loss in the 30-80° C. range attributed to the release of water molecules followed by a second loss in the 190-250° C. range, corresponding to the BS melting. Decomposition occurs at 360° C., as evidenced by the DTG curve. The DSC curve of the BS/β-CD inclusion compound exhibits one endothermic event at 58.7° C., but the strong peaks at 78.3° C. and 70.6° C. originally observable in the β-CD and in the physical mixture curves respectively are now absent, indicating the release of water molecules upon inclusion. In addition, the peak at 208.8° C. corresponds to the BS melting and finally that at 332.8° C. can be associated to a new thermal phenomenon of the supramolecular compound. Interestingly, the DSC curves of the BS/β-CD and BS/HP-β-CD inclusion compounds are very similar.

The XRD powder pattern diffraction analyses gave further support for the formation of a supramolecular compound between BS and β-CD. The XRD powder diffraction patterns of BS and β-CD exhibit sharp peaks, characteristic of crystalline compounds. The XRD pattern of the physical mixture shows peaks characteristic of BS and β-CD. In contrast, the BS/β-CD inclusion compound presents a pattern that suggests a loss of crystallinity with formation of a less organized system upon inclusion. Comparison of the XRD patterns of the BS/β-CD inclusion compound with that of the BS/HP-β-CD analogue, prepared previously, indicates that the latter is more amorphous and consequently more water soluble.

The signals in the NMR spectra of BS and β-CD were in agreement with data reported in the literature. Upon host-guest interaction, all hydrogen signals of BS shift to lower frequencies and the carbon signals to higher frequencies. Interestingly, the Cl, CH and C═O signals exhibit the most significant shifts upon interaction, confirming the inclusion of the BS molecule from the aryl ring to the carbonyl oxygen of the semicarbazone moiety into the β-CD cavity as ascertained by infrared data.

Changes were observed in all relaxation times but the most significant variations were obtained for the ring hydrogens, followed by N—H and C—H, in accordance with the 13C NMR and infrared results. It is worth noting that the minor T₁ change was observed for the NH2 hydrogens, suggesting that this group is less affected by host-guest interaction, probably due to its longer distance from the hydrophobic aryl ring and consequently from the β-CD cavity.

### EXAMPLE 3

### Comparison of the Anticonvulsant Effect of Free Benzaldehyde Semicarbazone (BS) and the BS/HP-β-CD and BS/β-CD Pharmaceutical Compositions in Wistar Normal Rats with Electroshock Induced Seizures (Maximum Electroshock Screening, MES)

Wistar rats from the main breeding stock of the Institute of Biological Sciences, Federal University of Minas Gerais, Brazil, maintained at the animal facilities of the Physiology Department, weighing 250-300 g, were used. They were kept at 24° C., in groups of 5 per cage receiving chow pellets and water ad libitum. The light/dark cycle was 12 h:12 h, with lights on at 7:00 am and lights off at 7:00 pm. Efforts were made in order to avoid any unnecessary distress to the animals, in accordance to the Guidelines for Animal Experimentation of Federal University of Minas Gerais, Brazil.

Electroshock seizures were induced by electric stimulus, produced by an ELEKTROSCHOCKGERAT apparatus (Karl Kolbe, Scientific Technical Supplies, Frankfurt, Germany) using a current of 70 mA, 60 Hz, during 1 second through a pair of ear clip electrodes.

The behavioral evaluation was carried out by analyzing the tonic component in a four points scale as follows: 0=no seizure; 1=forelimb extension without hind limb extension; 2=complete forelimb extension and partial hind limb extension; 3=complete hind limb extension, which stays parallel to the tail. To evaluate the effect of decreasing on electroshock induced seizures severity, it was taken as criteria the blockade of complete fore- and hind limb extension (score≦1).

In the MES model, BS blocked the hindlimb extension in about 90% of the animals (males) at 100 mg/Kg/ip and vo. The BS/HP-β-CD inclusion compound blocked completely the hindlimb extension at 35 mg/Kg/ip and vo in 100% of the animals and at 25 mg/Kg/ip in 67% of the animals. Rats were examined 30 and 240 minutes after administration of BS/HP-β-CD (vo). Whereas free BS exhibits no activity after 240 minutes, BS/HP-β-CD was active in 60% of the animals, indicating a more extended duration of the drug.

In the MES model of epilepsy the minimum dose necessary to produce anticonvulsant activity decreased from 100 mg/Kg (ip or vo) for the free semicarbazone to 25 mg/Kg/vo (75%) and 15 mg/Kg/ip (85%) for the BS/β-CD inclusion compound. Comparison with the results obtained previously by us for the BS/HP-β-CD inclusion compound, which allowed dose reduction of 75% ip and 65% vo reveals that the host-guest strategy that uses β-CD is even more effective. The reasons for this difference could be either the lower water solubility of the BS/β-CD inclusion compound as compared to the BS/HP-β-CD analogue or the β-CD greater adhesion to the mucous wall, which would allow a more extended duration of the drug.

In conclusion, taking into consideration that currently used drugs cause significant side effects, which may limit their maximal usefulness, the new strategy could be successfully employed in the preparation of pharmaceutical compositions of anticonvulsants.

### EXAMPLE 4

### Comparison of the Anticonvulsant Effect of Free Benzaldehyde Semicarbazone (BS) and the HP-β-CD/BS Pharmaceutical Composition in Wistar Audiogenic Rats (WAR) with Audiogenic Seizures (AS). Typically WARs Present Running Fits, Jumping, Atonic Falling Followed by Tonic-Clonic Seizures and Clonic Spasms when Submitted to High Intensity Sound Stimulus (120 dB SPL)

Female Wistar Audiogenic Rats (WARs) from our own inbred colony, maintained at the animal facilities of the Physiology Department of Federal University of Minas Gerais, Brazil, weighing 250-300 g, were used. They were kept at 24° C., in groups of 5 per cage receiving chow pellets and water ad libitum. The light/dark cycle was 12 h:12 h, with lights on at 7:00 am and lights off at 7:00 pm. Efforts were made in order to avoid any unnecessary distress to the animals, in accordance to the Guidelines for Animal Experimentation of Federal University of Minas Gerais, Brazil.

Audiogenic seizures (AS) were induced by a sound stimulus (120 dB) delivered into an acoustic chamber through a loud speaker, until tonic seizures appeared, or during a maximum of 1 minute. Behavior was evaluated by a severity index (SI) ranging from SI=0.0 to SI=1.0 (maximum).

Typically WARs present running fits, jumping and atonic falling followed by tonic-clonic seizures and clonic spasms (SI≧0.85). Animals were stimulated three times, once every three days before the beginning of experiments, in order to screen them for seizure severity (control recording). Seven days after the third stimulation they were used in the experiments. To evaluate the effect of decreasing on AS severity, it was taken as criteria the blockade of the tonic component of seizure, which means to obtain SI<0.61.

In the AS model, BS blocked the tonic component of seizures in 33, 50 and 83% of the animals at 50, 75 and 100 mg/Kg/ip respectively. The BS/HP-β-CD inclusion compound at 35 mg/Kg (vo and ip) blocked the tonic component of seizures in 100% of the animals.

In the AS model the minimum dose necessary to produce anticonvulsant activity decreased from 100 mg/Kg (vo and ip) for the free semicarbazone to 35 mg/Kg (vo and ip) for the BS/HP-β-CD inclusion compound, which represents' 65% of dose reduction. These results suggest that the host-guest strategy could be used in the preparation of new pharmaceutical compositions of anticonvulsant drugs.

In conclusion, taking into consideration that currently used drugs cause significant side effects, which may limit their maximal usefulness, the new strategy could be successfully employed in the preparation of pharmaceutical compositions of anticonvulsants.

### EXAMPLE 5

### Evaluation of the Effect of Semicarbazones and/or Thiosemicarbazones and/or Their Derivatives, as Non-Limiting Example Benzaldehyde Semicarbazone, on the Motor Activity of Mice

The effect of BS on the motor activity of mice was evaluated in order to investigate if inhibition of the nociceptive behavior in animals treated with BS would not be the result of a central depressor effect. The motor activity was evaluated in a rota-rod apparatus. During the experiment, the mice were placed on the rota-rod (14 rpm) and the time they spent in the apparatus was determined. The cut-off time was 1 min. After the basal lecture, the animals were treated with BS (10, 25 or 50 mg/kg, intraperitoneal; i.p.). The vehicle used was 25% dimethylsulphoxide (DMSO)+10% tween 80 in saline. Thirty minutes after the injection, the time spent in the rota-rod was determined again. BS did not reduce the time spent in the rota-rod. This result indicates that any BS inhibitory effect on the nociceptive response is unlikely to result from central depressor effect or motor incoordination.

### EXAMPLE 6

### Evaluation of the Effect of Semicarbazones and/or Thiosemicarbazones and Their Derivatives, as Non-Limiting Example Benzaldehyde Semicarbazone, on Nociception in Mice with the Licking Behavior Induced by Formaldehyde

In the model of licking behavior induced by formaldehyde, the inflammatory stimulus (0.92%, 20 μL) is injected into the dorsum of the right hindpaw of mice. Immediately after the injection, the animals present a nociceptive behaviour characterized by licking and biting the injected paw. The licking time is determined between 0 and 5 min (first phase) and 15 and 30 min (second phase) after formaldehyde injection. The first phase of the nociceptive response induced by formaldehyde in mice was inhibited by the highest dose of BS (50 mg/kg, i.p.). However, all doses of BS (10, 25 and 50 mg/kg, i.p-1 h) markedly inhibited the second phase of the nociceptive response in this model.

### EXAMPLE 7

### Evaluation of the Effect of Semicarbazones and/or Thiosemicarbazones and/or Their Derivatives, as Non-Limiting Example Benzaldehyde Semicarbazone on Nociception in Rats with Thermal Allodynia Induced by Carrageenan

BS also inhibited the nociceptive response in the thermal allodynia induced by carrageenan in Wistar male rats (200-250 g). In the model of thermal allodynia, the right hindpaw withdrawal latency to a thermal stimulus (Hargreaves apparatus—Ugo Basile, Italy) is determined in rats before the administration of any drug. The intensity of the thermal stimulus is adjusted to induce a basal latency about 12 s. After the basal lecture, carrageenan (1%, 50 μl) is injected into the right hindpaw. This inflammatory stimulus reduces the latency for paw withdrawal induced by the thermal stimulus, which is characterized as thermal allodynia. The previous treatment with BS (10, 25 ou 50 mg/kg, i.p., −1 h) statistically inhibited the nociceptive response in the thermal allodynia induced by carrageenan only at 50 mg/kg, i.p.

### EXAMPLE 8

### Evaluation of the Effect of Semicarbazones and/or Thiosemicarbazones and Their/or Derivatives, as Non-Limiting Example Benzaldehyde Semicarbazone on Carrageenan-Induced Edema in Rats

To evaluate if BS presents antiedematogenic activity, the model of carrageenan-induced edema in rats was used. In this model, before the administration of any drug, the right hindpaw volume is determined with a plethysmomether. After the division of the groups, carrageenan (1%, 50 μl) is injected into the plantar surface. At different times after the injection of carrageenan, the paw volume is determined again. BS (10, 25 ou 50 mg/kg, i.p., −1 h) inhibited the carrageenan-induced edema.

### EXAMPLE 9

### Evaluation of the Effect of Semicarbazones and/or Thiosemicarbazones and/or Their Derivatives, as Non-Limiting Example Benzaldehyde Semicarbazone on Nociception Using the Hot-Plate Model in Mice

In the hot-plate model, the mice are placed over a hot-plate (57° C.). The latency to jump or lick the paws is determined. In this model, direct and immediate activation of peripheral afferent fibers occurs. Thus, drugs that act mainly in the central nervous system inhibit the nociceptive response in this model. BS (10, 25 or 50 mg/kg, i.p., −1 h) did not exhibit anti-nociceptive effect in this model.

In conclusion, the results indicate that BS presents an antiinflammatory effect characterized by the inhibition of the nociceptive response induced by formaldehyde and the nociceptive response and edema induced by carrageenan. It is unlikely that the antinociceptive response induced by BS results from central depressor effect, motor incoordination or muscle relaxing effect. This conclusion is supported by the lack of effect in the rota-rod apparatus and also in the hot-plate model. BS presents a pharmacological profile that resembles that of anti-inflammatory drugs, a more marked inhibition of the second phase of the nociceptive response induced by formaldehyde, inhibition of the thermal allodynia and edema induced by carrageenan, but absence of effect- on the nociceptive response in the hot-plate model. However, the partial inhibition of the first phase of the nociceptive response induced by formaldehyde and our previous results demonstrating that BS presents anticonvulsant activity indicate that this drug present a wider pharmacological activities spectrum including both peripheral and central actions.

Therefore, the pharmaceutical compositions of the present invention are characterized by central and peripheral actions. Its is worth noting that a few drugs presents such profile. This indicates that semicarbazones and/or thiosemicarbazones and their derivatives and pharmaceutical compositions may be used to treat different pathological conditions. Another interesting aspect is the induction of central effects (anticonvulsant activity) without a marked central depressor effect or motor incoordination. This is suggestive that semicarbazones and/or thiosemicarbazones and their derivatives and pharmaceutical compositions may be useful to alleviate neuropathic pain, that is usually treated with centrally acting drugs.

Altogether, the results indicate that semicarbazones and/or thiosemicarbazones and their derivatives and pharmaceutical compositions may be useful to alleviate the pain and reduce the edema associated with many inflammatory conditions, as well as to alleviate the pain associated with neuropathic conditions.

### EXAMPLE 10

### Evaluation of the Effects of Semicarbazones and/or Thiosemicarbazones and/or Their Derivatives, and of Pharmaceutical Compositions with Cyclodextrins and/or Their Derivatives, as Non Limiting Examples Benzaldehyde Semicarbazone (BS) and the Pharmaceutical Compositions BS/HP-β-CD and BS/β-CD on the Angiogenesis Induced by Sponge Implant in Mice

The experiments were performed in male, Swiss mice (20-30 g; 12 weeks old) from the Animal House of the Institute of Biological Sciences, Federal University of Minas Gerais. Polyether-polyurethane sponge discs 5 mm thickness and 10 mm diameter with central cannula, used as a framework to induce fibrovascular tissue growth, were introduced through a skin incision in the back of mice anesthetized with 2,2,2-tribromoethanol (1 mg/kg) [Machado, R. D. P., Santos, R. A. S., Andrade, S. P., Life Sciences 66, 67, 2000]. The animals were housed individually and provided with normal food and water. Housing and anesthesia concurred with the guidelines established by our local Institutional Animal Welfare Committee.

Different groups of animals received intraperitoneal injections of BS in the doses of 0.05, 0.25, 0.5 and 2.5 mg/kg, 24 hours after implantation. Four more doses were given in the subsequent days. The animals were killed by cervical dislocation 9 days after implantation. The implants were removed and processed for hemoglobin determination (vascular index) using a modified Drabkin's method [Plunkett, M. L., Haley, J. A., Lab. Invest. 62, 510, 1990; Machado, R. D. P., Santos, R. A. S., Andrade, S. P., Life Sciences 66, 67, 2001]. One group of animals received injection of vehicle. The results showed that the compound inhibited the vascularization of the implants in a dose-dependent way. Thus, while the dose of 0.05 mg/kg was unable to reduce the hemoglobin content the other doses reduced the hemoglobin content in more than 50% as compared with the vehicle-treated group. This example shows the efficacy of the compound as an antiangiogenic agent.

The same protocol was used to test the efficacy of the bromobenzaldehyde semicarbazone (BrBs) in the dose of 0.25 mg/kg. The hemoglobin content increased in about 30% relative to the control group, showing the efficacy of this compound as pro-angiogenic compound.

The same protocol was used to test the efficacy of the 1:1 inclusion compounds between benzaldehyde semicarbazone and β-cyclodextrin BS/β-CD and hydroxyproyl-β-cyclodextrin BS/HP-β-CD in the dose of 0.05 mg/kg. The reduction in the hemoglobin content using the inclusion compounds was 55% and 45%, respectively, thus, similar to the inhibition obtained with the dose of 0.25 mg/kg of free BS. This result shows that the inclusion compounds are 5 fold more potent in inhibiting angiogenesis associated with the inflammatory process induced by the sponge implants.

1. A composition comprising semicarbazones, thiosemicarbazones, their derivatives, or mixtures thereof mixed with organo-aqueous or solid solutions of cyclodextrins or their derivatives selected from the group containing alkyl, hydroxialkyl, hydroxipropyl and acyl cyclodextrins with cross-linked cyclodextrins or cyclodextrin polymers and pharmaceutically acceptable carriers or excipients in solution or in the solid state

2. A composition comprising semicarbazones, thiosemicarbazones, their derivatives, or mixtures thereof mixed to pharmaceutically acceptable carriers or excipients in solution or in the solid state.

3. A composition according to with claim 1, wherein said composition is formulated for a therapeutic dose of 35 mg/Kg, when used as a 1:1 inclusion compound between benzaldehyde semicarbazone and hydroxipropyl-β-cyclodextrin.

4. A composition according to claim 1, wherein said composition is formulated for a therapeutic dose of 25 mg/Kg, when used as a 1:1 inclusion compound between benzaldehyde semicarbazone and β-cyclodextrin.

5. A composition according to claim 3 for use as an anticonvulsant.

6. A composition according to claim 4 for use as an anticonvulsant.

7. A product of semicarbazones, thiosemicarbazones, their derivatives, or mixtures thereof mixed with organo-aqueous or solid solutions of cyclodextrins comprising a composition as defined in claim 3.

8. A product of semicarbazones, thiosemicarbazones, their derivatives, or mixtures thereof mixed with organo-aqueous or solid solutions of cyclodextrins comprising a composition as defined in claim 4.

9. A product according to claim 7, for use as an anticonvulsant.

10. A product according to claim 8, for use as an anticonvulsant.

11. A composition according to claim 2 for use as anti-nociceptive agents.

12. A product of semicarbazones, thiosemicarbazones, their derivatives, or mixtures thereof comprising a pharmaceutical composition as defined in claim 2.

13. A product according to claim 12, for use as an anti-nociceptive agent.

14. A product according to claim 12, for use as an anti-inflammatory agent.

15. A product of semicarbazones, thiosemicarbazones, their derivatives, or mixtures thereof comprising a pharmaceutical composition as defined in claim 1, for use in angiogenic therapy.

16. A product of semicarbazones, thiosemicarbazones, their derivatives, or mixtures thereof comprising a pharmaceutical composition as defined in claim 2, for use in angiogenic therapy.

17. A product according to claim 7, formulated to be administered to humans and animals.

18. A product according to claim 8, formulated to be administered to humans and animals.

19. A product according to claim 7, in the form of tablets, capsules, granules, suppositories, sterile parenteral solutions or sterile parenteral suspensions, oral solutions or oral suspensions, and water-in-oil or oil-in-water emulsions.

20. A product according to claim 8, in the form of tablets, capsules, granules, suppositories, sterile parenteral solutions or sterile parenteral suspensions, oral solutions or oral suspensions, and water-in-oil or oil-in-water emulsions.
